(19) United States
(12) Patent Application Publication
(10) Pub. No.: US 2021/0275348 A1
(43) Pub. Date: Sep. 9, 2021

(54) Intravaginal Ring with Removal Device

(71) Applicant: Ellen M. Emas, Silver Spring, MD (US)

(72) Inventor: Ellen M. Emas, Silver Spring, MD (US)

(21) Appl. No.: 17/017,923

(22) Filed: Sep. 11, 2020

Related U.S. Application Data
(60) Provisional application No. 62/898,716, filed on Sep. 11, 2019.

(51) Int. Cl. A61F 6/12

(52) U.S. Cl. CPC A61F 6/12

(57) ABSTRACT

The outer peripheral surface of an intravaginal ring is modified in order to aid in the removal process of the intravaginal ring from the vagina. No foreign object, other than a fingertip, is needed in order to remove the intravaginal ring from the vaginal region.

### CROSS REFERENCE

This application claims priority from provisional application Ser. No. 62/898716 filed Sep. 11, 2019.

### FIELD OF THE INVENTION

The present invention is directed to modifying the shape of an intravaginal ring to improve ease of removal of the intravaginal ring from a vagina.

### BACKGROUND OF THE INVENTION

Intravaginal rings are used by women for a variety of purposes. One problem associated with these rings is the removal of the ring from the vaginal region. Difficulty in removing the ring occurs when the user can feel the ring with the user's fingertip but is unable to sufficiently engage the inner peripheral surface of the ring with enough of the fingertip in order to lower and remove the ring from the vagina.

US Patent Application Publication No. 20190029874 discloses a one-piece closed end finger cot with a substantially rigid hook which is inserted into the vagina and used to aid in the removal of the intravaginal ring.

US Patent Application Publication No. 20180193184 disclosed a tool which is inserted into the vagina to aid in the removal of the intravaginal ring.

Both US Patent Application Publications insert a foreign object into a vagina, which can cause injury or irritation as well as be uncomfortable to use by the user.

### SUMMARY OF THE INVENTION

The present invention is directed to modifying the outer peripheral surface of an intravaginal ring to aid in the removal process of the intravaginal ring from the vagina. Thus, no foreign object, other than a fingertip, is needed to remove the intravaginal ring from the vagina.

### DESCRIPTION OF THE DRAWINGS

FIG. 1 shows a top or bottom view of a first embodiment of an intravaginal ring,

FIG. 2 shows a top or bottom view of a second embodiment of an intravaginal ring,

FIG. 3 shows a top or bottom view of a third embodiment of an intravaginal ring,

FIGS. 4 shows a top or bottom view of a fourth embodiment of the intravaginal ring and FIG. 4a shows a variation for a removal device,

FIG. 5 shows a top or bottom view of a fifth embodiment of the intravaginal ring,

FIG. 6 shows a top or bottom view of a sixth embodiment of the intravaginal ring,

FIG. 7 shows a partial top or bottom view of a seventh embodiment of an intravaginal ring having an insertion ring and FIG. 7a shows a cross sectional view of the intravaginal ring with the insertion ring,

FIG. 8 shows a top or bottom view of the insertion ring for the intravaginal ring of the seventh embodiment,

FIG. 9 shows a top or bottom view of a variation of the insertion ring for the intravaginal ring of the seventh embodiment,

FIG. 10 shows a partial top or bottom view of an eighth embodiment of the intravaginal ring with an insertion ring and FIG. 10a shows a cross sectional view of the intravaginal ring with the insertion ring,

FIG. 11 shows a top or bottom of view of the insertion ring for the intravaginal ring of the eighth embodiment,

FIG. 12 shows a top or bottom view of a variation of the insertion ring for the intravaginal ring of the eighth embodiment,

FIG. 13 shows various arrangements of the removal tool on the intravaginal ring.

### DESCRIPTION OF THE INVENTION

The present invention is directed to modification of the shape of the outer peripheral surface of an intravaginal ring in order to ease removal of the intravaginal ring from the vaginal region. The present invention is not directed to the chemical or pharmacological composition of the intravaginal ring. The outer peripheral surface can be of the same chemical or pharmacological compositions or materials already in use for the intravaginal ring. Depending upon the shape of the removal device, the removal device may also serve as sexual assault deterrent device.

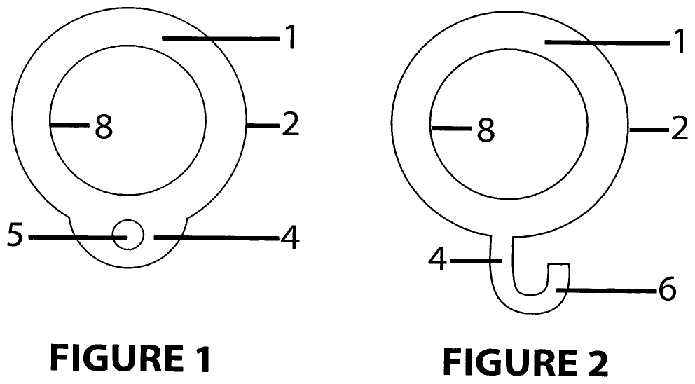

FIG. 1 shows a first embodiment of the present invention. As shown in FIG. 1, the outer peripheral surface 2 of the intravaginal ring 1 is modified to have a removal device 4. The removal device 4 is integrally formed as an extension of the outer peripheral surface 2 of the intravaginal ring 1. The removal device 4 maybe reinforced to have enough strength that it will not break when used to lower the intravaginal ring 1 in the vagina. As shown in FIG. 1, the removal device 4 is a projection, a protrusion or an extension of the outer peripheral surface having an orifice 5. The orifice 5 can be of any shape and size such as circular, oval, rectangular, triangular, etc. The projection, protrusion or extension of the removal device 4 can be of any shape, such a circular, oval, U-shape, V-shape, rectangular, semi-circular, etc., and can be of any size as long as the orifice 5 of the projection, protrusion or extension can be wholly or partially grasp by a fingertip. It should be noted that although the removal device 4 is shown in the drawings as extending from the outer circumferential surface, the removal device 4 can be provided as extending from anywhere on the outer peripheral surface of the intravaginal ring. Reference numeral 8 shown in FIG. 1 is the inner peripheral surface.

To remove the intravaginal ring 1, a fingertip is inserted into the vagina until it touches the intravaginal ring 1. If the intravaginal ring 1 is sufficiently within reach of the fingertip, the ring 1 can be removed in the normal fashion by looping the fingertip around the inner peripheral surface 8 of the ring 1 and pulling the ring 1 out of the vagina. Alternatively, if the removal device 4 is facing the lower part of the vagina, the fingertip can be wholly or partially inserted into the orifice 5 of the removal device 4. The fingertip can then use the orifice 5 of the removal device 4 to lower the ring sufficiently in the vagina so that the fingertip can grasp the inner peripheral surface 8 of the intravaginal ring 1 and remove it in the normal fashion or the fingertip can remove the intravaginal ring 1 from the vagina using just the removal device 4.

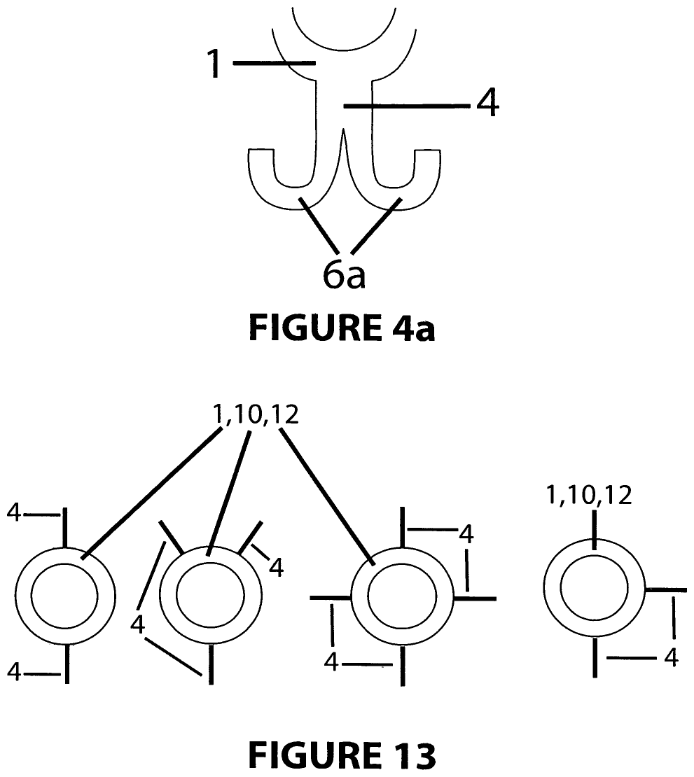

FIG. 2 shows a second embodiment. This embodiment has a similar outer peripheral surface 2 as the first embodiment except the removal device 4 is in a shape of a hook-like shape 6. The removal device 4 is integrally formed as an extension of the outer peripheral surface 2 of the intravaginal ring 1. The removal device 4 maybe reinforced to have enough strength that it will not break when used to lower the intravaginal ring 1 in the vagina. The hook-like shape 6 can be of any shape and is shown in a J-shape but can also be a C-shape, U-shape, L-shape or any other hook-like shape. In addition, the removal device 4 can be a double hook-like shape 6a as shown in FIG. 4a. The intravaginal ring 1 is removed as described above except instead of wholly or partially inserting a fingertip into an orifice 5, the fingertip instead wholly or partially grasps the hook-like shape 6 or 6a to lower or remove the intravaginal ring 1 from the vagina.

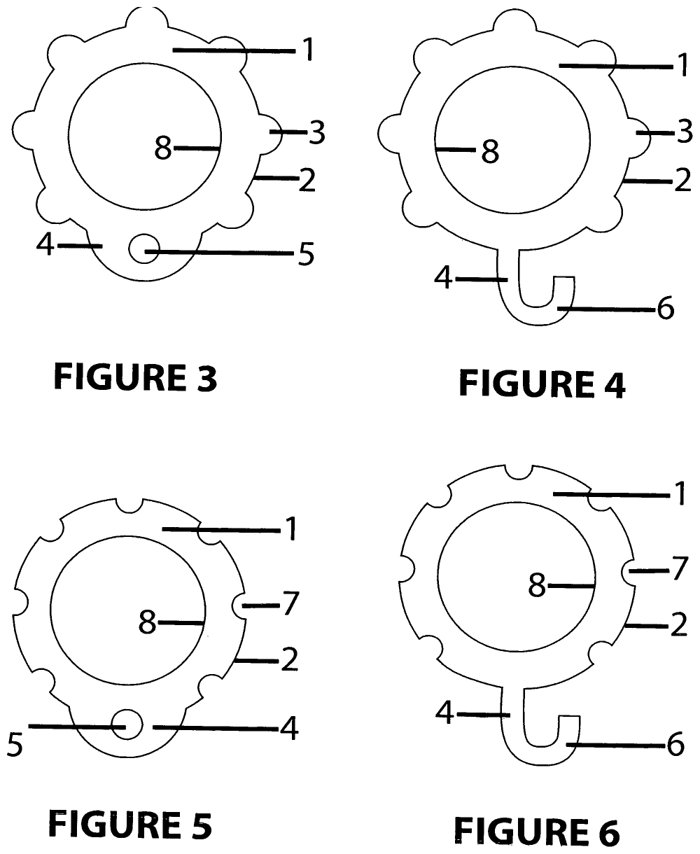

FIG. 3 shows a third embodiment of the present invention. In this embodiment the intravaginal ring 1 is formed with both a manipulation device 3 and a removal device 4. As shown in FIG. 3, the outer peripheral surface 2 of the intravaginal ring 1 is modified to have a manipulation device 3 formed as a plurality of projections, protrusions or extensions integrally formed as an extension of the outer peripheral surface 2 of the intravaginal ring 1. The plurality of projections, protrusion or extensions of the manipulation device 3 form an undulating outer peripheral surface 2. The undulating surface can have a regular or irregular placement of the projections, protrusions or extensions on the outer peripheral surface 2. The present invention should not be limited by the arrangement of the manipulation device 3 shown in the drawings and can be modified in shape, repetition and area provided on the outer peripheral surface. For example, FIG. 3 shows the intravaginal ring 1 formed with the removal device 4 formed in one area of the outer peripheral surface 2 and the manipulation device 3 formed over the entire remaining area of the outer peripheral surface 2. However, the manipulation device 3 may be formed in only a portion of the remaining area of the outer peripheral surface (i.e. not the entire remaining portion of the outer peripheral surface).

The plurality of projections, protrusions or extensions of the manipulation device 3 can be of any shape which allows a fingertip to manipulate the intravaginal ring 1 to rotate in the vagina. Thus, the shape of the projections, protrusions or extensions of the manipulation device 3 include such shapes as circular, oval, U-shape, V-shape, rectangular, semi-circular etc. The intravaginal ring 1 also has a removal device 4 integrally formed as an extension of the outer peripheral surface 2 as in the first embodiment. As shown in FIG. 3, the removal device 4 is a projection, a protrusion or an extension which has an orifice 5. The orifice 5 may be of any shape and any size as described above in the first embodiment as long as the orifice 5 can be wholly or partially grasp by a fingertip. The orifice 5 can be circular, oval, rectangular, etc. as described above.

To remove the intravaginal ring 1 of the third embodiment, a fingertip is inserted into the vagina until it touches the intravaginal ring 1. If the intravaginal ring 1 is sufficiently within reach of the fingertip, the ring 1 can be removed in the normal fashion by looping the fingertip around the inner peripheral surface 8 of the ring 1 and pulling the ring 1 out of the vagina. Alternatively, if the removal device 4 is facing the lower part of the vagina, the fingertip can be inserted into the orifice 5 of the removal device 4. The removal device 4 can then be used to lower the ring sufficiently in the vagina so that the fingertip can grasp the inner peripheral surface 8 of the intravaginal ring 1 and remove it in the normal fashion or the intravaginal ring 1 can be removed using just the removal device 4. However, if 1) the fingertip cannot grasp the inner peripheral surface 8 of the ring 1 and can only touch the outer peripheral surface 2 of the intravaginal ring 1, and 2) the removal device 4 has rotated away from the lower part of the vagina, the fingertip can rotate the intravaginal ring 1 in the vagina via the projections, protrusions or extensions of the manipulation device 3 until the removal device 4 is within reach of the fingertip (i.e. the fingertip is articulated to rotate the ring 1 as the fingertip moves from projection (3) to projection (3) thus rotating the ring 1 in the vagina via the manipulation device 3) . Once the removal device 4 is rotated to be within reach of the fingertip, the removal device 4 can be used, as described above, to lower the ring 1 sufficiently in the vagina to grasp the inner peripheral surface 8 of the intravaginal ring 1 and remove the ring 1 in the normal fashion or the intravaginal ring 1 can be removed using just the removal device 4, as described above.

FIG. 4 shows a fourth embodiment. This embodiment has a similar outer peripheral surface 2 with both a manipulation device 3 and a removal device 4 as in the third embodiment except the removal device 4 is in the shape of a hook-like shape 6 as in the second embodiment. The hook-like shape 6 can be of any shape and is shown in a J-shape but can also be a C-shape, U-shape, L-shape or any other hook-like shape as described above in the second embodiment. In addition, the removal device 4 can be a double hook-like shape 6a as shown in FIG. 4a. The intravaginal ring 1 is removed as described above in the third embodiment except instead of wholly or partially inserting the fingertip into an orifice 5, the fingertip instead wholly or partially grasps the hook-like shape 6 or 6a to lower the intravaginal ring 1 in the vagina in order to remove the ring 1 in the normal fashion or to remove the intravaginal ring 1 from the vagina using the hook-like shape 6 or 6a.

FIG. 5 shows a fifth embodiment. In this embodiment the plurality of projections, protrusions or extensions of the manipulation device 3 shown in FIGS. 3 and 4 are replaced by a manipulation device 7 formed of a plurality of indentations in the outer peripheral surface 2 thus forming an undulating outer peripheral surface 2. The undulating surface can have a regular or irregular placement of the indentations in the outer peripheral surface. The present invention should not be limited by the arrangement shown in the drawings and can be modified in shape, repetition and area of the outer peripheral surface. The plurality of indentations of the manipulation device 7 can be of any shape and size such as U-shape, V-shape, C-shape, semi-circular, semi-oval, semi-rectangular, etc. The outer peripheral surface 2 of the intravaginal ring 1 also has a removal device 4 with an orifice 5. The removal device 4 in the fifth embodiment is the same as the removal device 4 as described above in the first and third embodiments. If the removal device 4 of intravaginal ring 1 is not in the lower part of the vagina, a fingertip is inserted into one of the indentations 7 of the manipulation device 3. The fingertip is articulated to rotate the intravaginal ring 1 via the indentations until the next indentation of the manipulation device 7 is within reach. The fingertip continues to rotate the ring 1 until the removal device 4 is rotated to be in the lower part of the vagina so that the removal device 4 can be used to lower or remove the intravaginal ring 1 from the vagina as described above.

FIG. 6 shows a sixth embodiment having both 1) a manipulation device 7, as shown and described in the fifth embodiment and 2) a removal device 4. In this embodiment the removal device 4 is in the shape of a hook-like shape 6 or 6a as in the second and fourth embodiments shown in FIGS. 2, 4 and 4a described above. The intravaginal ring 1 is removed as described above in the fifth embodiment except instead of wholly or partially inserting the fingertip into an orifice 5, as in the fifth embodiment, the fingertip instead wholly or partially grasps the hook-like shape 6 or 6a in order to lower or remove the intravaginal ring 1 from the vagina.

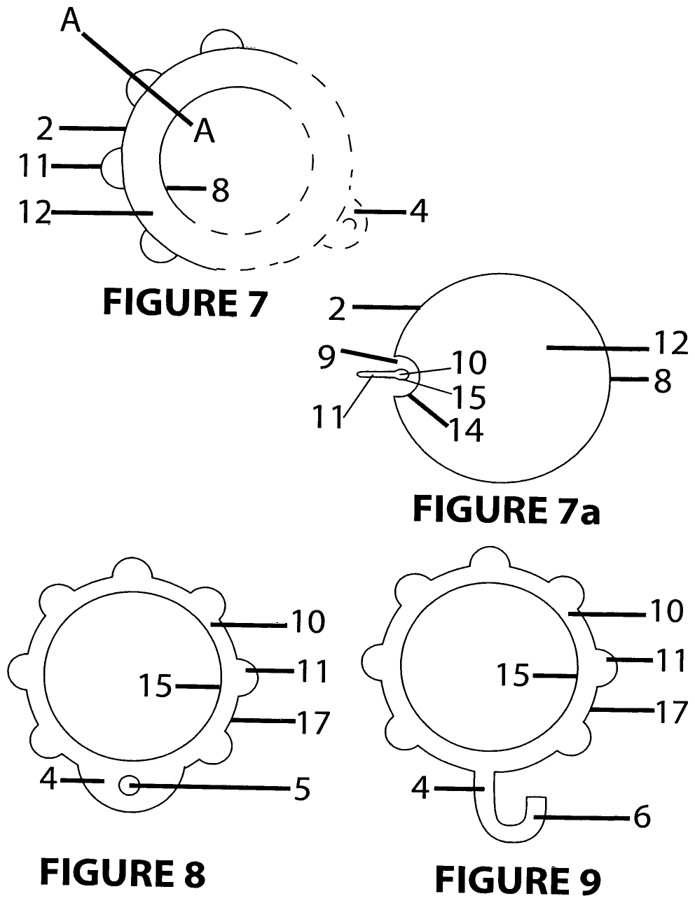

FIG. 7 show a partial top or bottom view of a seventh embodiment of an intravaginal ring 12 of the present invention. The intravaginal ring 12 is provided with one of the insertion rings 10 which is shown in FIG. 8 or 9. FIG. 7a shows a cross sectional view of FIG. 7 along line A-A. The intravaginal ring 12 shown in FIG. 7a has an inner peripheral surface 8 and an outer peripheral surface 2. The outer peripheral surface 2 is provided with a slot 9 which circumvents the intravaginal ring 12. The insertion ring 10 is provided inside the slot 9 and is rotatable inside the slot 9 of the intravaginal ring 12. The insertion ring 10 has an inner peripheral surface 15. The inner peripheral surface 15 is adjacent a surface 14 of the slot 9. Both the manipulation device 11 of the insertion ring 10 and the removal device 4 of the insertion ring 10 project outwardly from the slot 9. It should be noted that although the insertion ring 10 shown in FIGS. 7, 8 and 9 include manipulation device 11, the insertion ring 10 may only include the removal device 4. Thus, the insertion ring 10 will instead look like ring 1 shown in FIGS. 1 and 2. The insertion ring 10 may be of the same or different chemical or pharmacological compositions or materials as the intravaginal ring 12. In addition, the insertion ring 10 should be strong enough that it will not break when the inner peripheral surface 15 is pressed against slot 9 via the removal device 4 in order to lower the intravaginal ring 12 in the vagina for removal.

As shown in FIGS. 8 and 9, the insertion ring 10 has an inner peripheral surface 15 and an outer peripheral surface 17. The outer peripheral surface 17 of the insertion ring 10 of FIGS. 8 and 9 is provided with a plurality of projections, protrusions or extensions which form manipulation device 11. The projections, protrusions or extensions are of any shape and size as in the third and fourth embodiments described above. The outer peripheral surface is also provided with a removal device 4 as in the first through fifth embodiments. The removal device 4 in FIG. 8 is a protrusion or projection having an orifice 5 while the removal device 4 in FIG. 9 is a protrusion or projection having a hook-like shape 6. The hook-like shape 6 of the removal tool 4 may instead have a double hook-like shape 6a as shown in FIG. 4a.

In the third through sixth embodiments, the intravaginal ring 1 itself is rotated in the vagina in order to grasp the removal device 4 for removal. In the seventh embodiment the fingertip will instead rotate only the insertion ring 10 inside the slot 9 of the intravaginal ring 12 via the projections or protrusions of the manipulation device 11 until the removal device 4 of the insertion ring 10 is within reach of the fingertip. With the fingertip wholly or partially inserted into the removal device 4, by pulling on the removal device 4 the pressure of the insertion ring on the slot of the intravaginal ring 12 sufficiently lowers the intravaginal ring 12 in the vagina so that the fingertip can grasp the inner peripheral surface 8 of the intravaginal ring 12 and remove the ring 12 in the normal fashion or the fingertip can remove the intravaginal ring 12 using just the removal device 4. Alternatively, if the insertion ring 10 is not provided with the manipulation device 11, as in FIGS. 1 and 2, the intravaginal ring 12 is removed when the removal device 4 is located in the lower part of the vagina as described in the first and second embodiments.

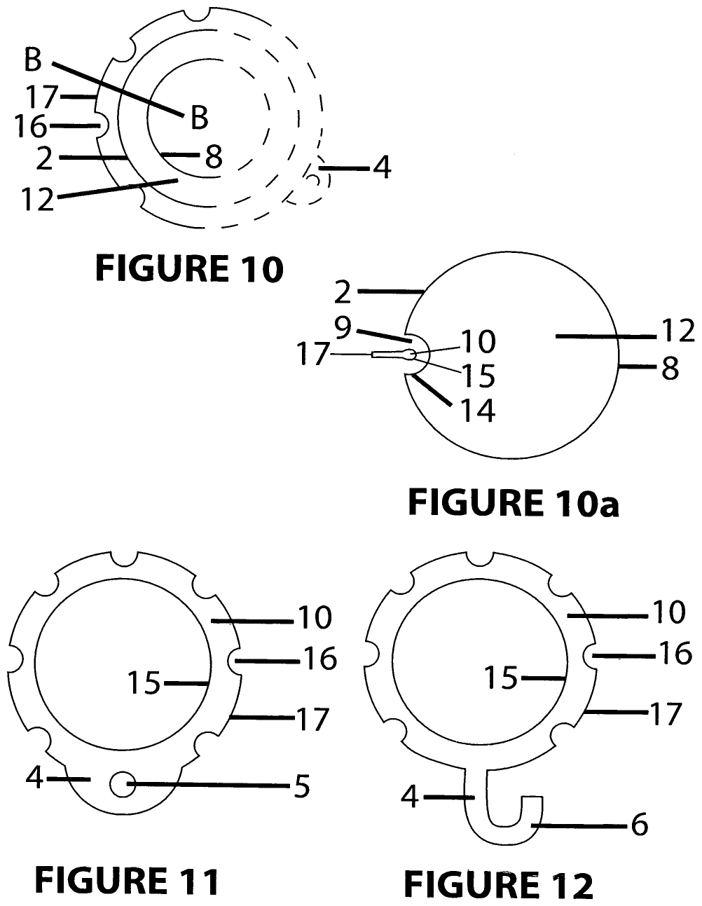

FIG. 10 show a partial top or bottom view of an eighth embodiment of an intravaginal ring 12. The intravaginal ring 12 is provided with one of the insertion rings 10 which is shown in FIG. 11 or 12. FIG. 10a shows a cross sectional view of FIG. 10 along line B-B. The intravaginal ring 12 has an inner peripheral surface 8 and an outer peripheral surface 2. As in the seventh embodiment, the outer peripheral surface 2 of intravaginal ring 12 is provided with a slot 9 which circumvents the ring 12. The insertion ring 10 of FIGS. 11 and 12 is provided inside the slot 9 and is rotatable inside the slot 9 of the intravaginal ring 12. The insertion ring 10 has an inner peripheral surface 15. The inner peripheral surface 15 is adjacent a surface 14 of the slot 9. Both the outer peripheral surface 17 of the insertion ring 10 and the removal device 4 of the insertion ring 10 project outwardly from the slot 9.

As shown in FIGS. 11 and 12, the insertion ring 10 has an inner peripheral surface 15 and an outer peripheral surface 17 provided with a plurality of indentations which form a manipulation device 16. The indentations are of any shape and size as in the fifth and sixth embodiments described above. The outer peripheral surface 17 is also provided with a removal device 4. The removal device 4 in FIG. 11 is a protrusion, projection or extension having an orifice 5 as in the first, third and fifth embodiments while the removal device 4 in FIG. 12 is a protrusion, projection or extension having a hook-like shape 6 as in the second, fourth and sixth embodiments. The hook-like shape 6 may instead have a double hook-like shape 6a as shown in FIG. 4a. It should be noted that although the insertion ring 10 shown in FIGS. 10, 11 and 12 include manipulation device 16, the insertion ring 10 may only include the removal device 4. Thus, the insertion ring 10 will instead look like ring 1 shown in FIGS. 1 and 2. The insertion ring 10 may be of the same or different chemical or pharmacological compositions or materials as the intravaginal ring 12. In addition, the insertion ring 10 should be strong enough that it will not break when the inner peripheral surface 15 is pressed against slot 9 via the removal device 4 in order to lower the intravaginal ring 12 in the vagina for removal.

In this embodiment, as in the seventh embodiment, instead of rotating the intravaginal ring 1 in the vagina as in the third to sixth embodiments, the user will instead rotate only the insertion ring 10 inside the slot 9 of the intravaginal ring 12 via inserting a fingertip into the indentations of the manipulation device 16 provided in the outer peripheral surface 17 of the insertions ring 10 until the removal device 4 is within reach of the fingertip. As described above, the fingertip can then use the removal device 4 to lower the intravaginal ring 12 sufficiently in the vagina to grasp the inner peripheral surface 8 of the intravaginal ring 12 and remove the intravaginal ring 12 in the normal fashion or the fingertip can remove the intravaginal ring 12 using just the removal device 4. Alternatively, if the insertion ring 10 is not provided with the manipulation device 16, as in FIGS. 1 and 2, the intravaginal ring 12 is removed when the removal device is located in the lower part of the vagina as described in the first and second embodiments.

In the above description the intervaginal rings 1 and 12 and insertion ring 10 are described as having one removal device 4. However, as shown in FIG. 13, the rings 1, 12 and 10 may be provided with multiple removal devices 4. It should be noted that the placement of the multiple removal devices in FIG. 13 is illustrative only and the invention is not limited thereto.

All of the various embodiments or options described herein can be combined in any and all variations. While the invention has been particularly shown and described with reference to some embodiments and drawings thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments and drawings but should be defined only in accordance with the following claims and their equivalents.

What is claimed is:

1). An intravaginal ring comprising:
an outer peripheral surface having at least one removal device as an extension thereof, the removal device a) projecting outwardly from the outer peripheral surface and b) integrally formed therewith, wherein the removal device aiding removal of the intravaginal ring from a vagina.

2). The intravaginal ring according to claim 1, wherein the at least one removal device is a protrusion having an orifice.

3). The intravaginal ring according to claim 1, wherein the at least one removal device is a protrusion having a hook-like shape.

4). The intravaginal ring according to claim 1, wherein the at least one removal device is a protrusion having a double hook-like shape.

5). The intravaginal ring according to claim 1, wherein more than one removal device is provided on the outer peripheral surface.

6). The intravaginal ring according to claim 1, wherein the outer peripheral surface further integrally provided with a manipulation device, formed as at least one of a) a plurality of projections and b) a plurality of indentations, the manipulation device manipulating a position of the intravaginal ring inside of the vagina.

7). An intravaginal ring comprising:
an outer peripheral surface having a) at least one removal device, as an extension of the outer peripheral surface, extending outwardly therefrom and integrally formed therewith, the removal device aiding removal of the intravaginal ring from a vagina and b) a manipulation device manipulation a position of the removal device inside of the vagina.

8). The intravaginal ring according to claim 7, wherein the at least one removal device is a projection having an orifice.

9). The intravaginal ring according to claim 7, wherein the at least one removal device is a projection having a hook-like shape.

10). The intravaginal ring according to claim 7, wherein the at least one removal device is a projection having a double hook-like shape.

11). The intravaginal ring according to claim 7, wherein more than one removal device is provided on the outer peripheral surface.

12). The intravaginal ring according to claim 7, wherein the manipulation device formed as at least one of 1) a plurality of protrusions and 2) a plurality of indentations.

13). An intravaginal ring comprising:
an outer peripheral surface provided with a slot; and
an insertion ring provided inside the slot and being rotatable within the slot, wherein the insertion ring having an outer peripheral surface integrally formed with at least one removal device, the removal device aiding removal of the intravaginal ring from a vagina, the outer peripheral surface of the insertion ring and the removal device projecting outwardly from the slot of the intravaginal ring.

14). The intravaginal ring according to claim 13, wherein the at least one removal device having an orifice.

15). The intravaginal ring according to claim 13, wherein the at least one removal device having a hook-like shape.

16). The intravaginal ring according to claim 13, wherein the at least one removal device is a protrusion having a double hook-like shape.

17). The intravaginal ring according to claim 13, wherein more than one removal device is provided on the outer peripheral surface of the insertion ring.

18), The intravaginal ring according to claim 13 wherein the outer peripheral surface of the insertion ring further comprising a manipulation device integrally formed on the outer peripheral surface of the insertion ring and projecting outwardly from the slot of the intravaginal ring, the manipulation device manipulating the insertion ring in the slot of the intravaginal ring.

19). The intravaginal ring according to claim 13, wherein the manipulation device formed as a plurality of protrusions.

20). The intravaginal ring according to claim 13, wherein the manipulation device formed as a plurality of indentations.
